(19) United States
(12) Patent Application Publication
(10) Pub. No.: US 2025/0268503 A1
(43) Pub. Date: Aug. 28, 2025

(54) CONDUCTIVE MATERIAL AND METHOD FOR MANUFACTURING THE SAME

(71) Applicant: AJOU UNIVERSITY INDUSTRY-ACADEMIC COOPERATION FOUNDATION, Suwon-si (KR)

(72) Inventor: Sungjun PARK, Yongin-si (KR)

(73) Assignee: AJOU UNIVERSITY INDUSTRY-ACADEMIC COOPERATION FOUNDATION, Suwon-si (KR)

(21) Appl. No.: 19/057,051

(22) Filed: Feb. 19, 2025

(30) Foreign Application Priority Data
Feb. 22, 2024 (KR) 10-2024-0025672
Oct. 28, 2024 (KR) 10-2024-0148684

(51) Int. Cl. A61B 5/268; A61B 5/27; D06M 11/83; D06M 13/148; D06M 15/356; D06M 15/63; G01N 27/30

(52) U.S. Cl. CPC A61B 5/268; A61B 5/27; D06M 11/83; D06M 13/148; D06M 15/3566; D06M 15/63; G01N 27/305; A61B 2562/125

(57) ABSTRACT

A conductive material according to an embodiment of the present disclosure comprises: a conductive polymer containing poly(3,4-ethylenedioxythiophene)-polystyrene sulfonic acid (PEDOT:PSS); a metal powder; a conductivity enhancing material containing ethylene glycol; and a stirring auxiliary material.

### CROSS-REFERENCE TO RELATED APPLICATION(S)

This application claims benefit of priority to Korean Patent Application No. 10-2024-0025672 filed on Feb. 22, 2024 and Korean Patent Application No. 10-2024-0148684, filed on Oct. 28, 2024 in the Korean Intellectual Property Office, the disclosure of which is incorporated herein by reference in its entirety.

### BACKGROUND

### 1. Field

The present disclosure relates to a conductive material and a method for manufacturing the same, and more particularly, to a conductive material having both high electrical properties and mechanical properties, and a method for manufacturing the same.

### 2. Description of Related Art

In order to manufacture a dry electrode or a dry conductive yarn, electrical or mechanical properties of a conductive material to be used as a material are considered.

Electrical properties are properties that indicate the degree to which a material carries current. They are related to parameters including resistivity, electrical conductivity, and current density, and serve as indicators for distinguishing types of materials. Conductive materials, including conductors and semiconductors, may be utilized in a variety of products and technical fields, including electronic devices and circuits.

Mechanical properties are properties that indicate the response or deformation of a material for forces and loads applied to the material. They are related to elongation, strength, elasticity, etc., and serve as a major factor in determining the final properties of the material, and it is important to use a material with mechanical properties suitable for the application field.

In particular, the development of materials having excellent electrical properties and mechanical properties such as high elongation has become increasingly important in order to manufacture devices with convenience and flexibility in a situation where research and development of body-wearing devices such as bio-signal measurement modules are being actively conducted.

To this end, research and development of various materials including polymers have been conducted, but it has been difficult to manufacture materials having high electrical and mechanical properties due to their incompatibility.

### SUMMARY

An object of the present disclosure is to solve the aforementioned problems and other problems related thereto.

An aspect of the present disclosure provides a material having both high elongation and conductivity and a method of manufacturing the same.

Another aspect of the present disclosure provides a method for manufacturing a dry electrode and a dry conductive yarn using a conductive material having excellent properties.

Aspects of the present disclosure are not restricted to those set forth above. Other aspects that are not mentioned above will be apparent to those of ordinary skill in the art from the following description.

According to an aspect of the present disclosure, a conductive material comprises a conductive polymer containing poly(3,4-ethylenedioxythiophene)-polystyrene sulfonic acid (PEDOT:PSS); a metal powder; a conductivity enhancing material containing ethylene glycol; and a stirring auxiliary material.

The stirring auxiliary material contained in the conductive material may contain any one or more of polyurethane and a surfactant.

The conductive material may contain an antioxidant.

In the conductive material, the conductive polymer may have a range of 2.5 to 6.5% by weight (wt %), the metal powder may have a range of 60 to 80% by weight, and the conductivity enhancing material may have a range of 0.1 to 0.4% by weight.

According to another aspect of the present disclosure, a method for manufacturing a conductive material comprises: preparing a first solution by adding a conductive polymer and a conductivity enhancing material; stirring the first solution; preparing a second solution by adding a metal powder and a stirring auxiliary material to the stirred first solution; and stirring the second solution, wherein the conductive polymer contains PEDOT:PSS and the conductivity enhancing material contains ethylene glycol.

The added stirring auxiliary material may contain any one or more of polyurethane or a surfactant.

The preparing of the second solution may comprise adding an antioxidant.

According to another aspect of the present disclosure, a method for manufacturing a dry electrode comprises: manufacturing the conductive material by any one of the methods described above; attaching the manufactured conductive material to a substrate; drying the attached conductive material to form a dry electrode; and detaching the formed dry electrode from the substrate.

According to another aspect of the present disclosure, a method for manufacturing a dry conductive yarn comprises: manufacturing the conductive material by any one of the methods described above; coating the manufactured conductive material onto a base fiber; and drying the coated conductive material to form the dry conductive yarn.

The coating of the conductive material may be performed by a syringe pump.

### BRIEF DESCRIPTION OF DRAWINGS

For a more complete understanding of the drawings cited in this specification, a brief description of each drawing is provided.

FIG. 1 is a diagram illustrating a process for manufacturing a conductive material according to an embodiment of the present disclosure.

FIG. 2 is a diagram illustrating a cross-section of a conductive material.

FIG. 3 is a diagram illustrating a method for manufacturing a conductive material.

FIG. 4 is a diagram illustrating an example of the properties of a conductive material.

FIG. 5 is a diagram illustrating another example of the properties of a conductive material.

FIG. 6 is a diagram illustrating another example of the properties of a conductive material.

### DETAILED DESCRIPTION

Since the technology disclosed herein may be modified in various ways and may have many embodiments, specific embodiments are illustrated in the drawings and described by the detailed description, wherein identical or similar components are given the same reference number regardless of drawing designation and duplicate descriptions are omitted. However, it is not intended to limit the technology disclosed herein to a specific embodiment, and it should be understood that the technology disclosed herein includes all modifications, equivalents, or substitutes that fall within in the spirit and scope of the technology disclosed herein.

In describing the technology disclosed herein, if it is determined that a detailed description of a related known technology may unnecessarily obscure the gist of the technology disclosed herein, the detailed descriptions will be omitted. In addition, numbers (e.g., first, second, etc.) used in the course of the description herein are merely identifiers to distinguish one component from another.

In addition, in this specification, when a component is referred to as being “connected” or “coupled” to another component, it should be understood that the component may be directly connected or directly coupled to the other component, but may also be connected or coupled through another component in between them, unless specifically indicated to the contrary. On the other hand, when a component is referred to as being “directly connected” or “directly coupled” to another component, it should be understood that there is no other component in between them.

In addition, in this specification, in the component expressed as “˜part”, two or more components may be combined into one component, or one component may be divided into two or more components based on more detailed functions. In addition, each of the components described below may additionally perform some or all of the functions of other components in addition to the primary functions for which it is responsible, and some of the primary functions for which it is responsible may be performed exclusively by other components.

The singular expression includes plural expression unless the context clearly indicates otherwise.

In the present application, it should be understood that the term “comprise” or “have” or the like are intended to specify the presence of a features, number, step, operations, component, part, or combinations thereof described in the specification, and are not intended to preclude in advance the possibility of presence or addition of one or more other features or numbers, steps, operations, components, parts, or combinations thereof.

Although the present disclosure has been illustrated and described in detail in the drawings and in the foregoing description, it will be understood that the present disclosure is to be considered illustrative rather than restrictive in character, and that only certain embodiments have been illustrated and described, and that all changes and modifications that fall within the spirit of the disclosure are desirable to be protected.

Hereinafter, embodiments according to the technical ideas of the technology disclosed herein will be sequentially described in detail.

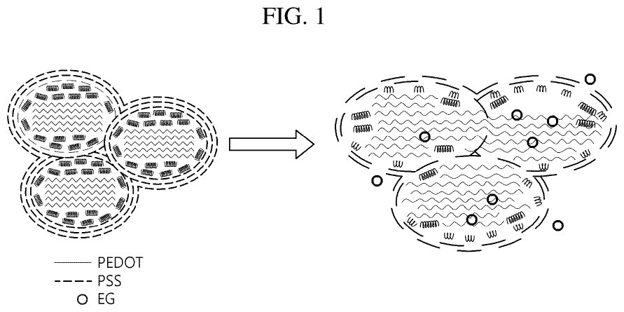

FIG. 1 is a diagram illustrating a process for manufacturing a conductive material containing a conductive polymer and a conductivity enhancing material, according to an embodiment of the present disclosure.

Referring to FIG. 1, the conductive polymer contains ply(3,4-ethylenedioxythiophene) (PEDOT) as shown as a solid line, and polystyrene sulfonic acid (PSS) as shown as a dotted line, and the conductivity enhancing material contains ethylene glycol (EG) as shown as a circle.

PEDOT is a polythiophene-based cationic polymer that has excellent conductivity, and thus may be utilized to manufacture transparent electrodes or high-conductivity materials.

PSS is a polystyrene-based anionic polymer that has both insulating and flexible properties, and thus may be utilized to manufacture wearable electronic devices or biomaterials.

In the present embodiment, PEDOT:PSS, a water-soluble polymer in which PEDOT and PSS are synthesized with each other, is used to manufacture a conductive material together with ethylene glycol.

Referring again to FIG. 1, a left side of FIG. 1 shows the form of a solution in which only PEDOT:PSS is present, and a right side of FIG. 1 shows a state in which ethylene glycol is added to a solution containing PEDOT:PSS.

Compared to the case where only PEDOT:PSS is present, it can be seen that when ethylene glycol is added, the structure of PEDOT and PSS and the boundary between each other become loose and natural stirring occurs. This is mainly because the addition of ethylene glycol to a PEDOT:PSS arrangement results in phase separation between the PEDOT and PSS chains along with a change in a PEDOT chain, which is attributed to electrical interaction, thereby improving the conductivity of the conductive material being manufactured.

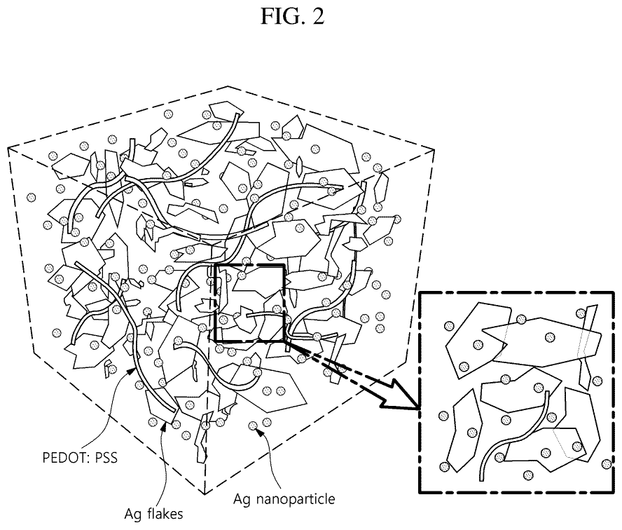

In addition, the conductive material according to the present embodiment contains a metal powder. Referring to FIG. 2, silver (Ag) powder may be used as an example of the metal powder.

The silver powder may be an inorganic material in the form of a powder that is pulverized to a size of 0.05 to 10.00 um. The silver powder has a high conductivity, and has a stable resistance value even at room temperature, and thus may be used to further improve the conductivity of the conductive material.

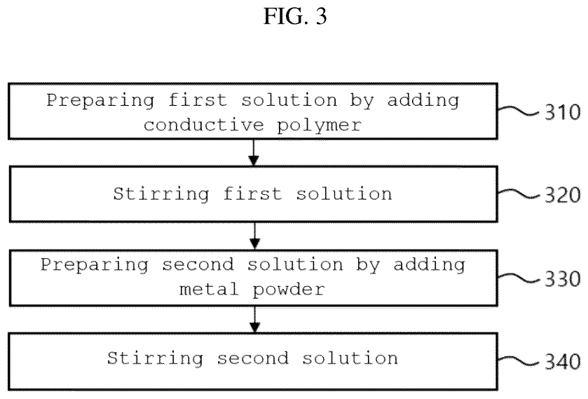

FIG. 3 is a diagram illustrating a method for manufacturing a conductive material according to an embodiment of the present disclosure.

In step 310, a first solution to which a conductive polymer is added is prepared.

The conductive polymer contains PEDOT:PSS, and a conductivity enhancing material containing ethylene glycol is added together to the first solution.

In step 320, the first solution prepared in step 310 is stirred.

The stirring may be performed using any apparatus and device, and in an example, the stirring may be performed for 5 to 10 minutes using a vortex mixer.

In step 330, a second solution in which a metal powder is added to the stirred first solution is prepared.

The metal powder may comprise a silver powder or a copper powder, and a stirring auxiliary material may be added to the second solution to assist in stirring of the components used in the manufacture of the conductive material together with the metal powder.

In an example, the stirring auxiliary material contains polyurethane, and the polyurethane in the form of a liquid has high elongation, so when stirred with the metal powder, enables the resulting material to have both good mechanical properties and strong adhesive properties.

In other example, the stirring auxiliary material contains a surfactant, and the surfactant in the form of a highly viscous liquid acts to aid in the agitation of hydrophilic and hydrophobic materials, resulting in more even agitation of the components in the solution.

Furthermore, an antioxidant may be added to the second solution as an auxiliary material to prevent oxidation of the metal powder.

In step 340, the previously prepared second solution is stirred.

The stirring may be performed using any apparatus and devices as in step 320, and in an example, the stirring of step 340 may be performed over several days.

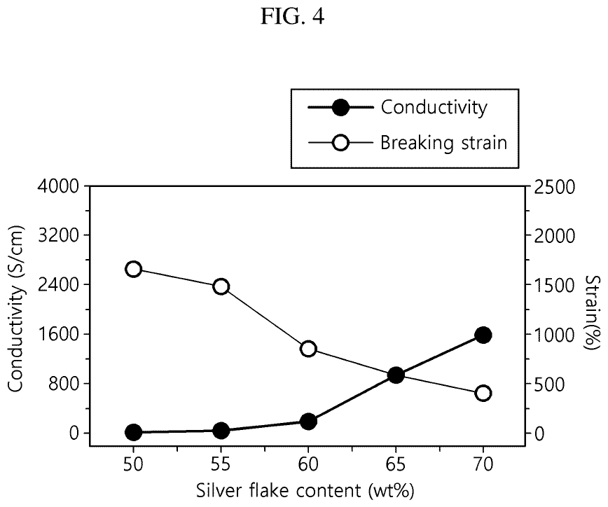

Now, refer to FIGS. 4 to 6 which illustrate the changes in the properties of the solution according to the ratio of the components contained.

FIG. 4 illustrates the properties of a solution containing a silver powder as an example of a metal powder.

In the embodiment illustrated in FIG. 4, the solution contains polyurethane and a surfactant as a stirring auxiliary material along with a silver powder, and a mixture of a silver powder and a surfactant is defined as silver flake. Here, a mixing ratio of a silver powder and a surfactant is set to 30:1.

It can be seen that as the ratio of silver flake increases, the electrical conductivity of the solution increase, such that the electrical properties are improved, whereas as the ratio of polyurethane decreases, the elongation, which is a mechanical property, decreases. Therefore, it may be desirable to use silver flake in the range of 60 to 80% by weight (wt %) to improve bot electrical and mechanical properties.

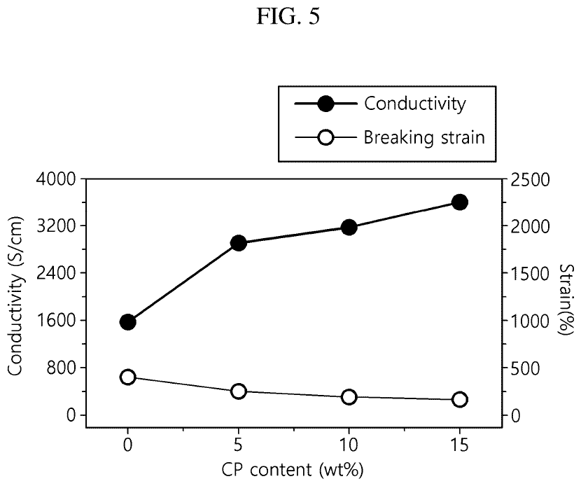

FIG. 5 illustrates the properties of a solution to which PEDOT:PSS and ethylene glycol are added in addition to the embodiment illustrated in FIG. 4.

In the present embodiment, a mixture of PEDOT:PSS as a conductive polymer, and ethylene glycol as a conductivity enhancing material, is defined as CP, and a mixing ratio of PEDOT:PSS and ethylene glycol is set to 20:1.

It can be seen that as the ratio of CP having high conductivity increases, the electrical conductivity of the solution increase, such that the electrical properties are improved, whereas the elongation decreases as a result of the low mechanical properties of CP. Therefore, it may be desirable to use a CP in the range of 3 to 7% by weight, which has both a high increase in electrical conductivity and a high elongation.

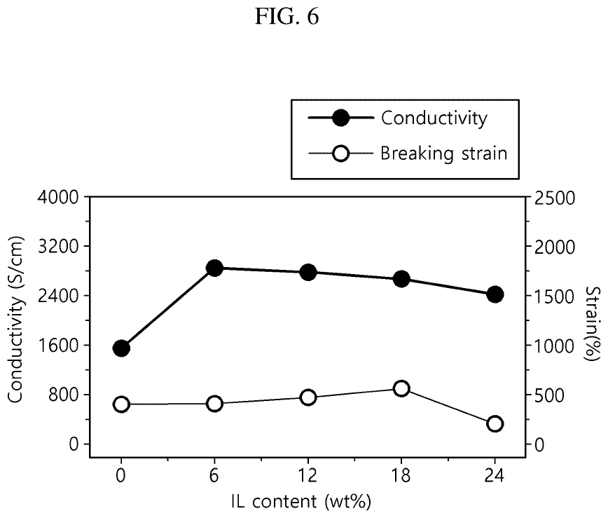

FIG. 6 illustrates the properties of a solution to which an ionic liquid (IL) is added in addition to the embodiment illustrated in FIG. 4.

In the present embodiment, it can be seen that EMIM⁺-TFSI⁻ type may be used as IL, and as the ratio of IL increases, the ratio of metal powder decreases, such that the electrical conductivity decreases, but IL forms a strong adhesive relationship with other components, such that elongation, which is a mechanical property, increases. Therefore, it may be desirable to use an IL in the range of 15 to 21% by weight, which has both a low decrease in electrical conductivity and a high elongation.

As a result, the conductive material according to the embodiment disclosed herein preferably contains 60 to 80% by weight of metal powder, 3 to 7% by weight of CP, and 15 to 21% by weight of IL, wherein since the ratio of PEDOT:PSS to ethylene glycol contained in the CP is 20:1, the PEDOT:PSS contained may have a range of about 2.5 to 6.5% by weight and the ethylene glycol contained may have a range of about 0.1 to 0.4% by weight.

Furthermore, the conductive material according to embodiment disclosed herein may be used to manufacture a dry electrode and a dry conductive yarn.

The dry electrode may be manufactured by attaching the manufactured conductive material to a substrate, drying the attached conductive material to form a dry electrode on the substrate, and detaching the formed dry electrode from the substrate.

Here, the conductive material may be attached to a substrate including a silicon substrate using a drop casting technique, and the drying of the conductive material may be performed at a temperature of 50 to 70° C. over a period of 3 to 5 hours.

The dry conductive yarn may be manufactured by coating the manufactured conductive material on the perimeter of a base fiber, and drying the coated conductive material to form a dry conductive yarn.

Here, the conductive material may be injected into a capillary of a syringe pump to coat the base fiber. The drying of the conductive material may likewise be performed at a temperature of 50 to 70° C. over a period of 3 to 5 hours.

The conductive material according to the present disclosure have both high elongation and conductivity, and thus may be used to manufacture electrodes and conductor yarns that require flexibility.

Furthermore, by presenting the weight ranges of components contained in the conductive material, it can be referenced to manufacture more optimized materials depending on the situation.

However, the effects that the technology can achieve are not limited to those mentioned above, and other effects that are not mentioned can be clearly understood by those skilled in the art from the description below.

The description described above presents the best mode of the technology disclosed herein, and provides examples to describe the technology disclosed herein and to enable those skilled in the art to make and use the technology disclosed herein. The specification thus prepared are not intended to limit the technology disclosed herein to the specific terms set forth.

Accordingly, while the technology disclosed herein has been described in detail with reference to the examples described above, modifications, alterations, and variations to the examples may be made by those skilled in the art without departing from the scope of the technology disclosed herein.

What is claimed is:

1. A conductive material comprising:
a conductive polymer containing poly(3,4-ethylenedioxythiophene)-polystyrene sulfonic acid (PEDOT:PSS);
a metal powder;
a conductivity enhancing material containing ethylene glycol; and
a stirring auxiliary material.

2. The conductive material of claim 1, wherein the stirring auxiliary material contains any one or more of polyurethane and a surfactant.

3. The conductive material of claim 1, further comprising an antioxidant.

4. The conductive material of claim 1, wherein the conductive polymer has a range of 2.5 to 6.5% by weight (wt %),
the metal powder has a range of 60 to 80% by weight, and
the conductivity enhancing material has a range of 0.1 to 0.4% by weight.

5. A method for manufacturing a conductive material, comprising:
preparing a first solution by adding a conductive polymer and a conductivity enhancing material;
stirring the first solution;
preparing a second solution by adding a metal powder and a stirring auxiliary material to the stirred first solution; and
stirring the second solution;
wherein the conductive polymer contains poly(3,4-ethylenedioxythiophene)-polystyrene sulfonic acid, and
the conductive enhancing material contains ethylene glycol.

6. The method of claim 5, wherein the stirring auxiliary material contains any one or more of polyurethane or a surfactant.

7. The method of claim 5, wherein the preparing of the second solution comprises adding an antioxidant.

8. A method for manufacturing a dry electrode, comprising:
manufacturing a conductive material by the method of claim 5;
attaching the manufactured conductive material to a substrate;
drying the attached conductive material to form a dry electrode; and
detaching the formed dry electrode from the substrate.

9. A method for manufacturing a dry conductive yarn, comprising:
manufacturing a conductive material by the method of claim 5;
coating the manufactured conductive material onto a base fiber; and
drying the coated conductive material to form the dry conductive yarn.

10. The method of claim 9, wherein the coating of the conductive material is performed by a syringe pump.
